(19) United States
(12) Patent Application Publication
(10) Pub. No.: US 2012/0271415 A1
(43) Pub. Date: Oct. 25, 2012

(54) TENDON FIXATION ANCHOR

(75) Inventors: Dirk Göthel, Vaihingen a. d.. Enz (DE); Hans Pässler, Heidelberg (DE)

(73) Assignee: RICHARD WOLF GMBH, KNITTLINGEN (DE)

(21) Appl. No.: 13/450,875

(22) Filed: Apr. 19, 2012

(30) Foreign Application Priority Data
Apr. 20, 2011 (DE) 10 2011 018 215.2

(51) Int. Cl. A61F 2/08

(52) U.S. Cl. 623/13.14

(57) ABSTRACT

A tendon fixation anchor is designed for being received in a bone channel and includes at least one first through-hole which extends transversely to an insertion direction of the anchor. The tendon fixation anchor can be inserted into a bone channel. A transplant tendon (TT) to be fastened can be led through the at least one first through-hole.

### BACKGROUND OF THE INVENTION

The invention relates to a tendon fixation anchor.

In the reconstruction of ligaments and tendons of the musculoskeletal system in the human or animal body, it is necessary to fasten the tendon transplant on the bone. This is particularly the case with the reconstruction of the front and rear cruciate ligaments in the knee joint. The criteria for an anatomically and functionally suitable fastening or fixation are a high resistance to being torn out, a fixation close to the joint and a minimization of the necessary tendon length. The minimization of the tendon length is therefore desirable since the necessary tendons are often taken from the body, whereby functional limitations at the related body parts occur. Inasmuch as this is concerned, it is desirable to remove as few tendons as possible.

### BRIEF SUMMARY OF THE INVENTION

With regard to these problems, it is an object of the invention to permit an improved fastening of tendons on the bone, which is secure and simultaneously renders possible a fixation close to the joint, with a minimized tendon length.

This object is achieved according to the present invention by a tendon fixation anchor which is designed for being received in a bone channel and comprises at least one first through-hole, which extends transversely to an insertion direction, in which the tendon fixation anchor can be inserted into a bone channel, through which first through-hole tendons to be fastened can be led.

Preferred embodiments of the invention are set forth in the subsequent description as well as the attached figures.

The tendon fixation anchor according to an embodiment of the invention is in particular suitable for the fastening of tendons in the reconstruction of the cruciate ligament, in particular for the fastening on the femoral side.

The tendon fixation anchor according to the invention is thus designed such that, for the fixation of a tendon, it can be inserted into a channel formed previously in the bone, i.e. in particular into a bore formed in the bone, and fixes the tendon in the inside of the bone channel. According to the invention, by way of the tendon fixation anchor, it is now rendered possible to carry out the fixation of the tendons in the bone, since the tendon fixation anchor is designed such that it can be inserted into the channel formed in the bone or into the bore formed in the bone. The required tendon length can now be shortened since the tendon can now be fixed directly in the bone (close to the joint).

The tendon fixation anchor according to the invention is designed such that it comprises a through-hole extending transversely to the insertion direction. The insertion direction is thereby the longitudinal direction of the bone channel or of the bore in the bone, into which channel or bore the tendon fixation anchor is inserted. The tendon fixation anchor with a retracted tendon is thereby pulled into the bone channel from one end. The first through-hole in the fixation anchor serves for fastening the tendon on the anchor. This is effected by way of the tendon being led through this through-hole before the insertion of the fixation anchor into the bone channel.

Subsequently, the tendon is pulled through the bone channel, and thus the fixation anchor is pulled into the bone channel. The tendon thereby preferably jams or clamps behind a projection formed in the bone channel or an abutment shoulder, which is formed in the bone channel and on which the fixation anchor comes to bear. This means that for this, the bone channel is designed such that it has a cross-sectional change, i.e. a region with a greater cross section, which receives the fixation anchor, and a connecting region with a smaller cross section, through which the tendon then extends further through the bone. Due to the fact that the tendon is led through the through-hole, it can be turned over once on the fixation anchor and thus embraces or wraps the fixation anchor.

Preferably, the tendon fixation anchor is designed as one part or in a single-piece manner. Moreover, it usefully comprises no moving parts. Preferably, the tendon fixation anchor comprises a second through-hole which is designed for receiving a suture for fastening a tendon pulled through the first through-hole. The tendon can be sewn on the fixation anchor by way of this second through-hole, for example by a suture being led through the second hole and around an outer side of the fixation anchor and simultaneously being led through the tendon. Thereby, the seam does not need to resist the tensions forces which occur later in the joint, since the tendon with the fixation anchor is clamped or jammed in the bone channel, but serves mainly for fixing the tendon on the fixation anchor on inserting the fixation anchor into the bone channel.

The second through-hole extends preferably parallel to the first through-hole. Thus the tendon can be led along via the second through-hole, so that a suture led through the second through-hole can extend transversely to the extension direction of the tendon through this tendon.

Further preferably, the second through-hole has a smaller, in particular significantly smaller, diameter than the first through-hole. The second through-hole only needs to be so large in diameter that a suture can be led once or several times through this hole, while the first through-hole must be so large that a tendon or a complete tendon bundle, which is to be fastened as a transplant on the bone, can be led through this through-hole. This means the first through-hole with regard to its diameter is selected such that it can receive the tendon transplant.

The second through-hole in the insertion direction preferably lies in front of the first through-hole. The insertion of the tendon fixation anchor into the bone channel is preferably effected in a manner such that the tendon is pulled through the bone channel, wherein the fixation anchor is previously fastened on the end of the tendon, and then with the tendon is pulled into the bone channel, where it then jams and/or comes to bear on a projection in the bone channel and thus fixes the tendon. If the second through-hole is situated in front of the first through-hole in the insertion direction, then the tendon which is led through the first through-hole extends past the second through-hole, so that a suture can be led through the second through-hole transversely through the tendon, in order to fasten this on the fixation anchor.

The tendon fixation anchor according to the invention further preferably comprises two diametrically opposed, first peripheral surface sections which are designed as bearing-contact surfaces for bearing on the peripheral wall of a bone channel. In particular, these first peripheral surface sections are preferably shaped such that they can come to bear on the inner wall of the bone channel or of a bore formed in the bone, in a large-surfaced manner. For this, the peripheral surface sections preferably have a shape, in particular a curvature which corresponds to the shape or curvature of the inner wall of the bone channel. The first through-hole, seen in the insertion direction, preferably lies in an axial section of the fixation anchor in which the peripheral surface sections, which come to bear on the bone, are formed.

For this purpose, the first peripheral surface sections are further preferably curved about a common longitudinal axis extending in the insertion direction. This means the peripheral surface sections seen in cross section form sections of a circle extending about the longitudinal axis. Here, the radius of curvature is preferably selected such that it corresponds essentially to the inner diameter of the bone channel, into which the tendon fixation anchor is to be inserted, or is slightly smaller than the radius of the bone channel. Since the bone channel, as described above, is usually formed by way of drilling, it usually has a circular cross section. With the described design of the peripheral surface sections, these can thus come to bear on the circular inner surface of the bone channel. The two peripheral surface sections thereby preferably form the outer peripheral sections of the fixation anchor, i.e. those peripheral surfaces which have the maximal outer diameter.

The tendon fixation anchor is fixed in the bone channel transversely to the insertion direction in the bone channel, preferably without play, by way of the bearing contact of these peripheral surface sections on the inner surfaces of the bone channel. Since the tendon fixation anchor further preferably has a greater length in the insertion direction than in the diameter direction between the two first peripheral surface sections, moreover, one prevents the tendon fixation anchor from being able to rotate in the bone channel about an axis extending transversely to the insertion direction.

Further preferably, two second peripheral surface sections, which are diametrically opposite one another, are situated between the mentioned first peripheral surface sections in the peripheral direction. These second peripheral surface sections thus in the peripheral direction connect the first peripheral sections to one another. The first through-hole and, optionally, the second through-hole extend between these second peripheral sections in the diametric direction with respect to the longitudinal axis of the fixation anchor, i.e. its insertion direction.

Thus, the tendon to be fastened is preferably led via these second peripheral surface sections in a direction parallel to the insertion direction or longitudinal axis of the fixation anchor and then led through the first through-hole and led back again on the opposite, second peripheral surface sections, so that this tendon, led through the first through-hole, wraps the fixation anchor. On inserting the fixation anchor into the bone channel, the tendon can then be clamped between the second peripheral surface sections and the inner wall of the bone channel.

The second peripheral surface sections are preferably designed in a planar manner. Thus, seen in a cross-sectional plane, they extend essentially in a chord-like manner to the arcs formed by the first peripheral surface sections. Thus, further preferably, the diametric distance between the two second peripheral surface sections is smaller than the diametric distance between the two first peripheral surface sections. Since as described, the bone channel is usually designed with a circular cross section, in each case a free space remains between the second peripheral surface sections and the oppositely lying inner wall of the bone channel, on inserting the fixation anchor into the bone channel. The tendon can be led through this free space.

The free space however is preferably dimensioned such that, with a fixation anchor inserted completely into the bone channel, the tendon is clamped between the second peripheral surface of the fixation anchor and the surrounding wall of the bone channel. This means that the diametrical distance between the second peripheral surface sections is preferably selected such that it is smaller than the diameter of the bone channel by an amount which is smaller than double the thickness of the tendon transplant to be received.

Particularly preferably, the tendon fixation anchor is designed such that its diameter tapers in the insertion direction. This is particularly the diameter between the previously described first peripheral surface sections. This means that these first peripheral surface sections thus form sections of a cone. The fixation anchor thus tapers in a cone-shaped manner in the insertion direction. This simplifies the introduction into the bone channel.

Moreover, a secure jamming in the bone channel can be achieved, in particular if the bone channel is conically shaped in the corresponding direction. Thus, the distance between the peripheral surfaces of the fixation anchor, in particular the first peripheral sections of the fixation anchor and the inner peripheral surfaces of the bone channel, can reduce on inserting the fixation anchor into the bone channel, until the fixation anchor comes to bear on the inner periphery of the bone channel preferably without play, and is thus securely clamped in the inside of the bone channel.

The tendon fixation anchor according to the invention permits a new type of method for the fixation of a tendon with the reconstruction of ligaments and tendons of the musculoskeletal system, in particular for fastening a tendon transplant with cruciate ligament reconstruction.

First, a bone channel or a bore is introduced into the bone, in which the tendon transplant is to be fastened. This bone is preferably the femur. The bone channel is thus formed by way of special instruments such that it obtains a bottle-neck shape. This means that the bone channel comprises at least two sections with a differently large cross section or diameter, wherein the section with the smaller cross section or diameter faces the joint. The section with the larger cross section or diameter is designed for receiving a tendon fixation anchor according to the preceding description.

In particular, the bone channel in this region has a shape and a diameter which are matched to the tendon fixation anchor or its outer shape, such that the tendon fixation anchor can clamp itself into the bone channel. Thereby, it comes to bear in particular on the bearing-contact shoulder which is formed by the cross-sectional reduction, on the inner periphery of the bone channel and is thus fixed in the bone. This contact shoulder in the bone channel is thus designed such that it is situated as close as possible to the joint, but the remaining web on the inner periphery of the bone channel in the extension direction of the bone channel has such a thickness that it is adequately stable, in order to be able to accommodate the forces exerted by the fixation anchor.

Next, the transplant is led by a loop through the first through-hole of the fixation anchor and then is fixed by a holding suture, which is led through the second through-hole, on the tendon fixation anchor. The holding suture thereby extends preferably on both sides of the fixation anchor in each case through the region of the tendon which is situated there, and then in a spaced manner, once again through the tendon, so that it then runs on the outer side of the fixation anchor to its diametrically opposite side. This means that the holding suture wraps round the fixation anchor.

Subsequently, the tendon fixation anchor together with the tendon transplant which is fastened thereon is then inserted into the bone channel in the later pulling direction which forms the insertion direction. The tendon fixation anchor then clamps itself or jams close to the entry of the bone channel to the joint, on the bearing-contact shoulder which is formed there by the cross-sectional reduction. The tendon transplant is also clamped between this bearing-contact shoulder or this web and the fixation anchor.

Thus, this remaining bony (cortical) web on the inner periphery of the bone channel, at the exit opening of the bone channel which faces the joint, serves as a barrier to the joint side and ensures an extremely tear-resistant connection in the loading direction of the transplant. The transplant length can be shortened by way of this fastening of the tendon transplant which is very close to the joint, compared to a fastening on the rear side of the bone.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a schematic lateral view of a knee joint;

FIG. 2 is a top perspective view of a fixation anchor according to an embodiment of the invention;

FIG. 3 is a lateral view of the fixation anchor according to FIG. 2; and

FIG. 4 is a sectional view of a bone channel with the inserted fixation anchor.

### DETAILED DESCRIPTION OF THE INVENTION

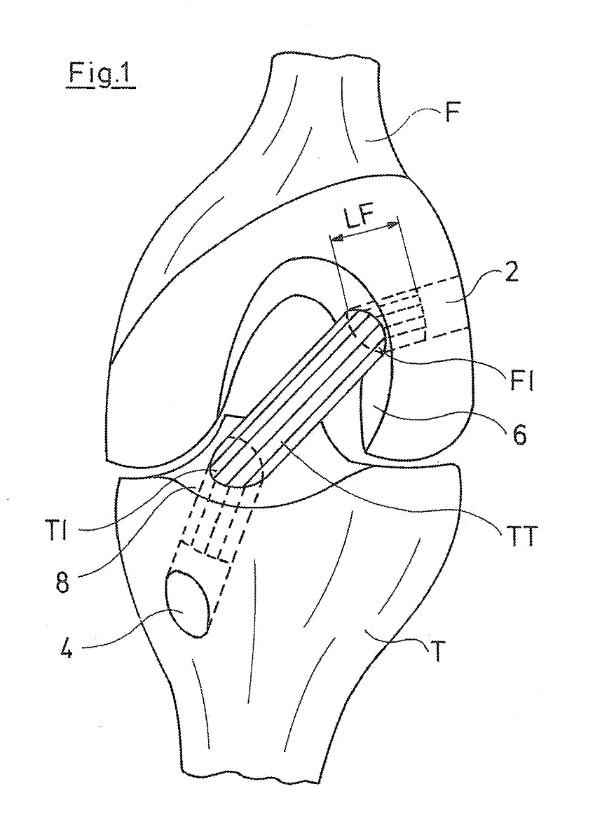

FIG. 1 schematically shows the knee joint with a tibia T and femur F, between which a cruciate ligament (tendon) transplant TT is arranged. The tendon transplant TT extends with its femoral end FI into a bone channel 2 in the femur and with its tibial end TI into a bone channel 4 in the tibia T. The bone channels 2 and 4 each extend from the joint sides 6 and 8 of the femur and tibia, respectively, to the respective rear sides of the bone. This means that they are designed as through-channels or through-holes.

In the shown embodiment, the fastening of the tendon transplant TT in the bone channel 2 in the femur is effected with the help of a fixation anchor 10, which is inserted into the inside of the bone channel 2. In this manner, the tendon transplant TT at its femoral end FI does not need to extend through the complete bone channel, but has a necessary length LF in the bone channel 2, which is shorter than the bone channel 2.

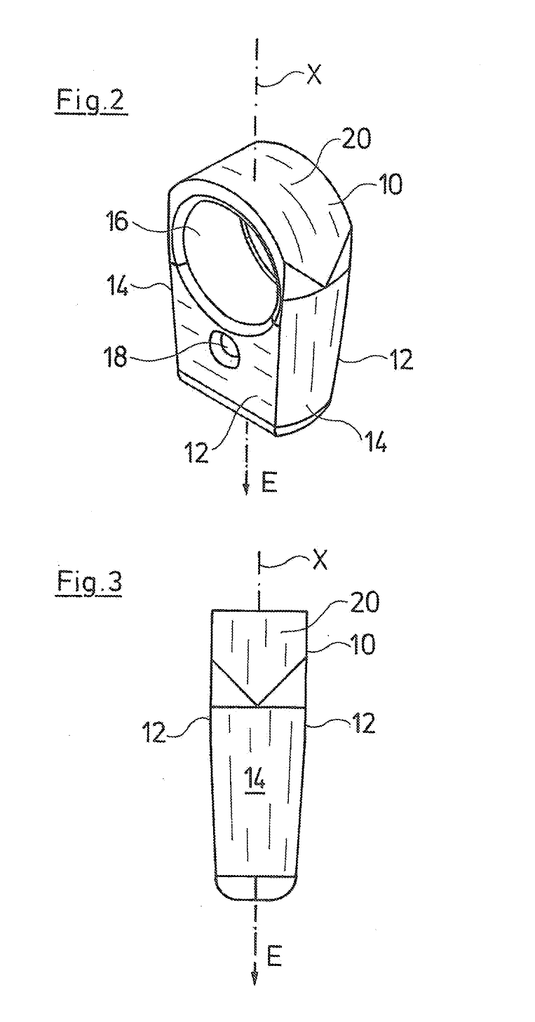

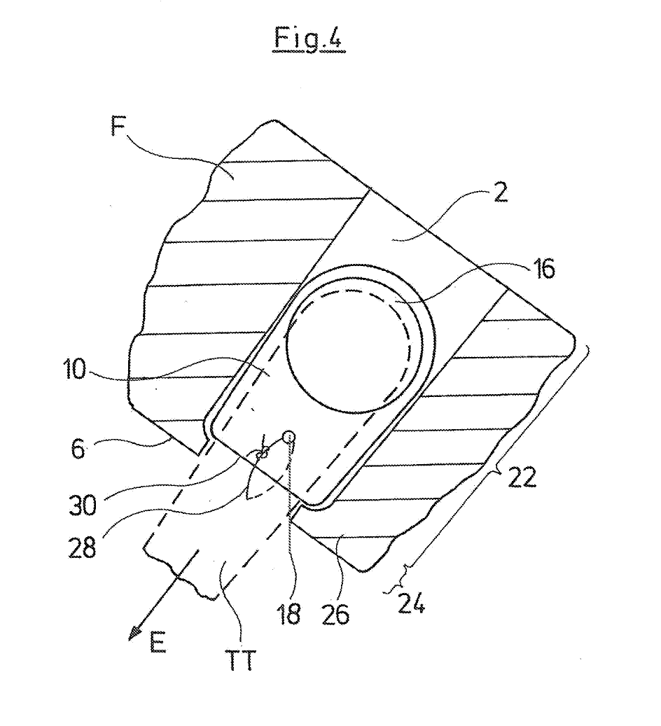

FIGS. 2 and 3 show the fixation anchor in detail. The fixation anchor 10 is designed as a cone with two flattened, diametrically oppositely lying peripheral surface sections 12. These peripheral surface sections 12, as second peripheral surface sections, lie between the two peripheral surface sections 14 which are arranged in a diametrically opposite manner. The first peripheral surface sections 14 form sections of the outer peripheral surface of a cone and are curved about the longitudinal axis X of the fixation anchor 10. The diameter or radius of curvature reduces in the insertion direction E, which runs in the direction of the longitudinal axis X of the fixation anchor 10.

The fixation anchor 10 comprises a first through-hole 16 extending normally to the longitudinal axis X between the second peripheral surface sections 12. The through-hole 16 is designed for receiving the transplant TT and has a diameter which is selected so large that the tendon transplant TT can extend through the through-hole 16. The first through-hole 16 has a maximal diameter which is slightly smaller than the maximum distance of the first peripheral surface sections 14 in the diametric direction. Preferably, the diameter of the through-hole 16 is larger than the minimal distance of the first peripheral surface sections 14 in the diametric direction, i.e. larger than the smallest diameter of the fixation anchor 10 between the two first peripheral surface sections 14 at the tapered end of the cone. Thus, the diameter of the through-hole 16 can be just as large or larger than the smallest diameter of the bone channel 2.

The diameter of the bone channel 2 can also be designed such that it is adapted to the thickness of the tendon bundle or tendon transplant TT which is to be received, i.e. the bone channel 2 only needs to be designed insignificantly larger than the thickness of the tendon transplant TT, so that it does not lead to a weakening of the bone.

A second through-hole 18 extends between the second peripheral surface sections 12, in a manner parallel to the first through-hole 16 and spaced from it in the insertion direction E. This second through-hole serves for receiving a suture for the fixation of the tendon transplant TT on the fixation anchor 10.

FIG. 4 shows that the end 20 of the fixation anchor 10, which is at the rear in the insertion direction E, is designed in a curved manner, wherein the curved surface of the rear end 20 extends essentially concentrically to the middle axis of the first through-hole 16.

As is to be seen in FIG. 4, the bone channel 2 in the femur is designed in the manner of a bottle neck. This means that the bone channel 2 has a first section 22 with a larger diameter and a second section 24 with a smaller diameter, wherein the second section 24 is adjacent the joint side 6 of the femur F. The second section 24 with the smaller diameter thus forms a cross-sectional reduction with a bearing-contact shoulder 26. The second section 24 in the extension direction of the bone channel 2 is designed in a very short manner compared to the first section 22, so that it merely forms a web which projects radially inwardly form the peripheral wall of the bone channel 2 and is adjacent the joint side 6, i.e. on the end of the bone channel 2 which faces the joint. The inner cross section or the inner diameter of the section 24 is smaller than the smallest diameter of the fixation anchor 10 between the first peripheral surface sections 14 defining the cone surfaces. The greatest distance or diameter of the fixation anchor 10 between the first peripheral surface sections 14, as is to be seen in FIG. 4, corresponds essentially to the inner diameter of the first section 22 of the bone channel 2.

Before inserting the fixation anchor 10 into the bone channel 2, the tendon transplant TT is led in the manner of a loop through the first through-hole 16 of the fixation anchor 10, so that the tendon transplant TT bears on the two second peripheral surface sections 12 of the fixation anchor 10. There, the tendon transplant TT is fixed with the help of a suture 28 which is led through the second through-hole 18 of the fixation anchor 10 and around its side 30 which is at the front in the insertion direction.

Subsequently, the tendon is pulled in the insertion direction E through the bone channel 2 and thus moves the end of the tendon transplant TT with the fixation anchor 10 from the side which is away from the joint, into the bone channel 2, until the fixation anchor 10 comes to bear in a clamping manner on the bearing-contact shoulder 26, which is defined by the section 24 which is smaller in diameter. Then the tendon transplant TT is also clamped between the fixation anchor 10 and the inner wall of the bone channel 2, in this region, so that a secure fixation is achieved here.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A tendon fixation anchor (10) designed to be received in a bone channel (2), the tendon fixation anchor comprising an anchor body having an insertion direction (E) in which the tendon fixation anchor (10) is inserted into the bone channel (2) and at least one first through-hole (16) extending through the anchor body transversely to the insertion direction (E), wherein a tendon transplant (TT) to be fastened is led through the at least one first through-hole (16).

2. The tendon fixation anchor according to claim 1, further comprising at least one second through-hole (18) extending transversely through the anchor body and designed to receive a suture (28) for fastening a tendon transplant (TT) led through the at least one first through-hole (16).

3. The tendon fixation anchor according to claim 2, wherein the at least one second through-hole (18) extends parallel to the at least one first through-hole (16).

4. The tendon fixation anchor according to claim 2, wherein the at least one second through-hole (18) has a smaller diameter than the at least one first through-hole (16).

5. The tendon fixation anchor according to claim 2, wherein the at least one second through-hole (18) is situated in front of the at least one first through-hole (16) in the insertion direction (E).

6. The tendon fixation anchor according to claim 1, wherein the anchor body comprises two diametrically opposite, first peripheral surface sections (14) which serve as bearing-contact surfaces for bearing on a peripheral wall of the bone channel (2).

7. The tendon fixation anchor according to claim 6, wherein the first peripheral surface sections (14) are curved about a common longitudinal axis (X) extending in the insertion direction (E).

8. The tendon fixation anchor according to claim 6, wherein the anchor body further comprises two diametrically opposite second peripheral surface sections (12) situated in a peripheral direction between the two first peripheral surface sections (14), and wherein the at least one first through-hole (16) extends between the two second peripheral surface sections (12).

9. The tendon fixation anchor according to claim 8, wherein the at least one second through-hole (18) extends between the two second peripheral surface sections (12).

10. The tendon fixation anchor according to claim 8, wherein the two second peripheral surface sections (12) have a planar peripheral surface.

11. The tendon fixation anchor according to claim 7, wherein a diametric distance between the two second peripheral surface sections (12) is smaller than a diametric distance between the two first peripheral surface sections (14).

12. The tendon fixation anchor according to claim 1, wherein the anchor body tapers in diameter in the insertion direction (E).

13. A method for fixation of a tendon transplant in a reconstruction of ligaments and tendons of a musculoskeletal system, the method comprising the following steps:
forming a bone channel in a bone in which the tendon transplant is to be fastened, wherein the bone channel has a bottle-neck shape defining a bearing-contact shoulder inside the bone channel,
leading the tendon transplant in a looping manner through a through hole of a tendon fixation anchor, and
inserting the tendon fixation anchor together with the looped tendon transplant into the bone channel, such that the tendon fixation anchor clamps on the bearing-contact shoulder.
